(19) United States
(12) Patent Application Publication
(10) Pub. No.: US 2026/0249027 A1
(43) Pub. Date: Aug. 27, 2026

(54) METERED DOSE INHALERS AND HIGH-DOSE SUSPENSIONS

(71) Applicant: Kindeva Drug Delivery L.P., Woodbury, MN (US)

(72) Inventors: Benjamin Myatt, Leicester (GB); Philip Cocks, West Bridgford (GB); Sarah Wrigglesworth, Loughborough (GB); Alexander Slowey, Stapleford (GB)

(21) Appl. No.: 19/160,140

(22) PCT Filed: Feb. 28, 2023
(86) PCT No.: PCT/US2023/014154

§ 371 Date: Aug. 27, 2025

(51) Int. Cl. A61M 15/00; A61K 31/135; A61K 31/352; A61K 31/496; A61K 31/58; A61K 47/06; A61M 11/00; A61M 11/02

(52) U.S. Cl. CPC A61M 15/0065; A61K 31/135; A61K 31/352; A61K 31/496; A61K 31/58; A61K 47/06; A61M 11/001; A61M 11/02; A61M 2202/0007; A61M 2202/06; A61M 2205/3379

(57) ABSTRACT

A metered dose inhaler comprising: a metering valve; a canister; and an actuator comprising an actuator nozzle; wherein the canister comprises a formulation, the formulation comprising greater than 70% by weight of HFO-1234ze(E); and at least one active pharmaceutical ingredient suspended in the formulation; wherein the metered dose inhaler delivers at least 0.5 milligram (mg) of the at least one active pharmaceutical ingredient per actuation. A metered dose inhaler comprising: a metering valve; a canister; and an actuator comprising an actuator nozzle; wherein the canister comprises a formulation, the formulation comprising greater than 70% by weight of HF A-152a; and at least one active pharmaceutical ingredient suspended in the formulation; wherein the metered dose inhaler delivers at least 0.5 milligram (mg) of the at least one active pharmaceutical ingredient per actuation.

The present application claims priority to U.S. Provisional Application No. 63/315,337, filed Mar. 1, 2022, which is incorporated herein by reference in its entirety.

### BACKGROUND

Delivery of aerosolized medicament to the respiratory tract for the treatment of respiratory and other diseases can be done using, by way of example, pressurized metered dose inhalers (pMDI), dry powder inhalers (DPI), or nebulizers. pMDIs are familiar to many patients who suffer from asthma or chronic obstructive pulmonary disease (COPD). pMDI devices can include an aluminum canister, sealed with a metering valve, that contains medicament formulation. Generally, a typical current medicament formulation includes one or more medicinal compounds present in a liquefied hydrofluoroalkane (HFA) propellant.

Historically, the propellants in most pMDIs have been chlorofluorocarbons (CFCs). However, environmental concerns during the 1990s led to the replacement of CFCs with hydrofluoroalkanes (HFAs) as the most commonly used propellant in pMDIs. Although HFAs do not cause ozone depletion, they do have a stated high global warming potential (GWP), which is a measurement of the future radiative effect of an emission of a substance relative to that of the same amount of carbon dioxide (CO₂). The two HFA propellants most commonly used in pMDIs are HFA-134a, also called HFC-134a, R-134a, or norflurane (CF₃CH₂F, 1,1,1,2-tetrafluoroethane) and HFA-227, also called HFC-227, FM-200, or apaflurane (CF₃CHFCHF₃, 1,1,1,2,3,3,3-heptafluoropropane) having stated 100-year GWP values of 1300 to 1430 and 3220 to 3350, respectively.

Various other propellants have been proposed over the years. Among them, hydrofluoroolefins (HFOs) and carbon dioxide (CO₂) have been mentioned as potential propellants for pMDIs, but no pMDI product has been successfully developed or commercialized using either as a propellant. It has now been found that HFA-152a, also called HFC-152a, DFE, or R-152a (C₂H₄F₂, 1,1-difluoroethane) and HFO-1234ze(E), also called R-1234ze (C₃H₂F₄, (1E)-1,3,3,3-tetrafluoropropene) can be used as pMDI propellants. One advantage of such pMDIs is their low stated GWP.

### SUMMARY

In one embodiment, a pMDI (also referred to herein as an MDI or metered dose inhaler) is provided that includes: a metering valve; a canister; and an actuator that includes an actuator nozzle; wherein the canister includes a formulation (i.e., composition), the formulation including greater than 70% by weight of HFO-1234ze(E), and at least one active pharmaceutical ingredient suspended in the formulation; wherein the metered dose inhaler delivers at least 0.5 milligram (mg) of the at least one active pharmaceutical ingredient per actuation.

In one embodiment, a metered dose inhaler is provided that includes: a metering valve; a canister; and an actuator that includes an actuator nozzle; wherein the canister includes a formulation, the formulation including greater than 70% by weight of HFA-152a, and at least one active pharmaceutical ingredient suspended in the formulation; wherein the metered dose inhaler delivers at least 0.5 milligram (mg) of the at least one active pharmaceutical ingredient per actuation.

Herein, the term “comprises” and variations thereof do not have a limiting meaning where these terms appear in the description and claims. Such terms will be understood to imply the inclusion of a stated step or element, or group of steps or elements, but not the exclusion of any other step or element, or group of steps or elements. The phrase “consisting of” means including, and limited to, whatever follows the phrase “consisting of.” Thus, the phrase “consisting of” indicates that the listed elements are required or mandatory, and that no other elements may be present. The phrase “consisting essentially of” means including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may, or may not, be present depending upon whether or not they materially affect the activity or action of the listed elements. Any of the elements or combinations of elements that are recited in this specification in open-ended language (e.g., comprise and derivatives thereof), are considered to additionally be recited in closed-ended language (e.g., consist and derivatives thereof) and in partially closed-ended language (e.g., consist essentially and derivatives thereof).

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the disclosure.

Throughout this disclosure, singular forms such as “a,” “an,” and “the” are often used for convenience; singular forms are meant to include the plural unless the singular alone is explicitly specified or is clearly indicated by the context.

As used herein, the term “or” is generally employed in its usual sense including “and/or” unless the content clearly dictates otherwise.

The term “and/or” means one or all of the listed elements or a combination of any two or more of the listed elements.

The phrase “ambient conditions” as used herein, refers to an environment of room temperature (approximately 20° C. to 25° C.) and 30% to 60% relative humidity.

Also herein, all numbers are assumed to be modified by the term “about” and in certain embodiments, preferably, by the term “exactly.” As used herein in connection with a measured quantity, the term “about” refers to that variation in the measured quantity as would be expected by the skilled artisan making the measurement and exercising a level of care commensurate with the objective of the measurement and the precision of the measuring equipment used. Herein, “up to” a number (e.g., up to 50) includes the number (e.g., 50). Herein, “at least” a number (e.g., at least 50) includes the number (e.g., 50). Herein, “no more than” a number (e.g., no more than 50) includes the number (e.g., 50).

Numerical ranges, for example “between x and y” or “from x to y,” include the endpoint values of x and y. Also herein, the recitations of numerical ranges by endpoints include all numbers subsumed within that range as well as the endpoints (e.g., 1 to 5 includes 1, 1.5, 2, 2.75, 3, 3.80, 4, 5, etc.).

Some terms used in this application have special meanings, as defined herein. All other terms will be known to the skilled artisan and are to be afforded the meaning that a person of skill in the art at the time of the invention would have given them.

Elements in this specification that are referred to as “common,” “commonly used,” “conventional,” “typical,” “typically,” and the like, should be understood to be common within the context of the compositions, articles, such as inhalers and pMDIs, and methods of this disclosure; this terminology is not used to mean that these features are present, much less common, in the prior art. Unless otherwise specified, only the Background section of this Application refers to the prior art.

Reference throughout this specification to “one embodiment,” “an embodiment,” “certain embodiments,” “one or more embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments.

The present disclosure will be described with respect to embodiments and with reference to certain drawings, but the invention is not limited thereto. The drawings described are only schematic and are non-limiting. In the drawings, the size of some of the elements can be exaggerated and not drawn to scale for illustrative purposes.

The above summary of the present disclosure is not intended to describe each disclosed embodiment or every implementation of the present disclosure. The description that follows more particularly exemplifies illustrative embodiments. In several places throughout the disclosure, guidance is provided through lists of examples, which examples may be used in various combinations. In each instance, the recited list serves only as a representative group and should not be interpreted as an exclusive or exhaustive list. Thus, the scope of the present disclosure should not be limited to the specific illustrative structures described herein, but rather extends at least to the structures described by the language of the claims, and the equivalents of those structures. Any of the elements that are positively recited in this specification as alternatives may be explicitly included in the claims or excluded from the claims, in any combination as desired. Although various theories and possible mechanisms may have been discussed herein, in no event should such discussions serve to limit the claimable subject matter.

The complete disclosure of all patents, patent applications, publications, and electronically available material cited herein are incorporated by reference in their entirety. In the event that any inconsistency exists between the present disclosure and the disclosure(s) of any document incorporated herein by reference, the present disclosure shall govern. The detailed description and examples herein have been given for clarity of understanding only. No unnecessary limitations are to be understood therefrom. The invention is not limited to the exact details shown and described, for variations obvious to one skilled in the art will be included within the invention defined by the claims. All headings are for the convenience of the reader and should not be used to limit the meaning of the text that follows the heading, unless so specified.

### BRIEF DESCRIPTION OF THE DRAWINGS

The present disclosure will be described with respect to embodiments and with reference to certain drawings, but the invention is not limited thereto. The drawings described are only schematic and are non-limiting. In the drawings, the size of some of the elements can be exaggerated and not drawn to scale for illustrative purposes.

FIG. 1 is a cross-sectional side view of an inhaler including a canister containing a valve according to the present disclosure.

FIG. 2 is a detailed cross-sectional side view of the inhaler of FIG. 1.

FIG. 3 is a cross-sectional side view of a metering valve for an inhaler.

### DETAILED DESCRIPTION

The formulations of the present disclosure are suspensions (i.e., suspension formulations or suspension compositions). That is, the formulations include one or more active pharmaceutical ingredients (APIs) dispersed in the formulations (e.g., suspended in the propellant) to form suspensions. Herein, in a “suspension” the API is in a microparticulate solid form (typically micronized, but can also be size-reduced by a multitude of other particle size reduction techniques (e.g. ball or jet milling, spray drying, freeze drying, spray freeze drying, high-pressure homogenization, supercritical fluid technology, controlled crystallization, sonocrystallization, wet polishing, etc.) and dispersed in a propellant, optionally with other soluble or non-solubilized excipients to aid the suspension behavior of the particles. Herein, a suspension is a dispersion of particles of particulate material (e.g., API) that is visible to the unaided human eye, although there may also be a small amount of solubilized particulate material within the composition. For suspension formulations, solubilization of an API is generally undesirable. In embodiments, it may be desirable to minimize solubilization of an API.

Solution and suspension formulations are fundamentally different pMDI formulation approaches. Different factors need to be considered when undertaking the development of products using either of these formulation approaches. Accordingly, it is not possible to apply the same knowledge and understanding of solution formulations to suspension formulations. Suspensions, for example, need to achieve a degree of physical stability to avoid significant separation of the physical mixture via sedimentation or creaming of the suspended particles. This can lead to poor dose consistency over time. Therefore, for suspensions, suspension aids are often used to control flocculation. Also, in suspensions, the resultant aerosol particle size is predominantly influenced by the geometric particle size of the microparticulate API that can change if the API particles are partially soluble in the propellant/formulation, which can lead to physical instability over time, through particle growth. In suspensions, the aerosol particle size is influenced by the size and geometric shape of the microparticulate API used to for the suspension, which can change if the API dissolves in the formulation. Dissolved API particles may grow over time, leading to physical instability of the formulation and changes to product performance. Inhalers including suspension formulations often have problems associated with deposition of the suspended API particles on the internal surfaces of the canister and valve, which again can cause changes to product performance over time. These problems are specific to suspensions and any teachings specific to solutions do not necessarily overcome them.

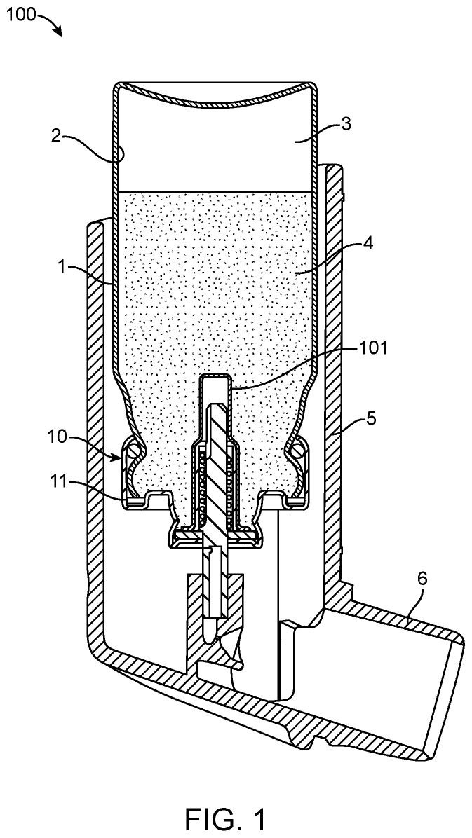

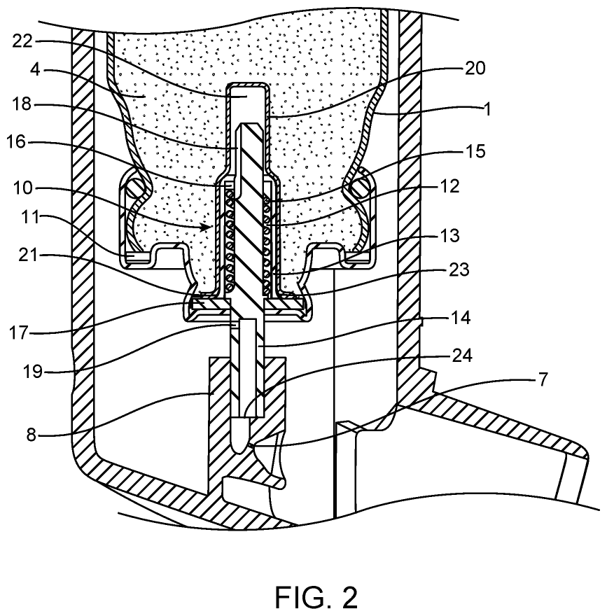

The various embodiments of formulations described herein can be utilized with any suitable inhaler. For example, FIG. 1 shows one embodiment of a metered dose inhaler 100, including an aerosol canister 1 fitted with a metered dose metering valve 10 (shown in its resting position). The metering valve 10 is typically affixed, i.e., crimped, onto the canister via a cap or ferrule 11 (typically made of aluminum or an aluminum alloy) which is generally provided as part of the valve assembly. Between the canister and the ferrule there may be one or more seals. In the embodiments shown in FIGS. 1 and 2 between the canister 1 and the ferrule 11 there are two seals including, e.g., an O-ring seal and a gasket seal.

As shown in FIG. 1, the canister/valve dispenser is typically provided with an actuator 5 including an appropriate patient port 6, such as a mouthpiece. For administration to the nasal cavities the patient port is generally provided in an appropriate form (e.g., smaller diameter tube, often sloping upwardly) for delivery through the nose. Actuators are generally made of a plastic material, for example polypropylene or polyethylene. As can be seen from FIG. 1, inner walls 2 of the canister and outer walls 101 of the portion(s) of the metering valve 10 located within the canister define a formulation chamber 3 in which aerosol formulation 4 is contained.

The valve 10 shown in FIGS. 1 and 2, includes a metering chamber 12, defined in part by an inner valve body 13, through which a valve stem 14 passes. The valve stem 14, which is biased outwardly by a compression spring 15, is in sliding sealing engagement with an inner tank seal 16 and an outer diaphragm seal 17. The valve 10 also includes a second valve body 20 in the form of a bottle emptier. The inner valve body 13 (also referred to as the “primary” valve body) defines in part the metering chamber 12. The second valve body 20 (also referred to as the “secondary” valve body) defines in part a pre-metering region or chamber besides serving as a bottle emptier.

Referring to FIG. 2, aerosol formulation 4 can pass from the formulation chamber 3 into a pre-metering chamber 22 provided between the secondary valve body 20 and the primary valve body 13 through an annular space 21 between a flange 23 of the secondary valve body 20 and the primary valve body 13. To actuate (fire) the valve 10, the valve stem 14 is pushed inwardly relative to the canister 1 from its resting position shown in FIGS. 1 and 2, allowing formulation to pass from the metering chamber 12 through a side hole 19 in the valve stem and through a stem outlet 24 to an actuator nozzle 7 then out to the patient. When the valve stem 14 is released, formulation enters into the valve 10, in particular into the pre-metering chamber 22, through the annular space 21 and thence from the pre-metering chamber through a groove 18 in the valve stem past the tank seal 16 into the metering chamber 12.

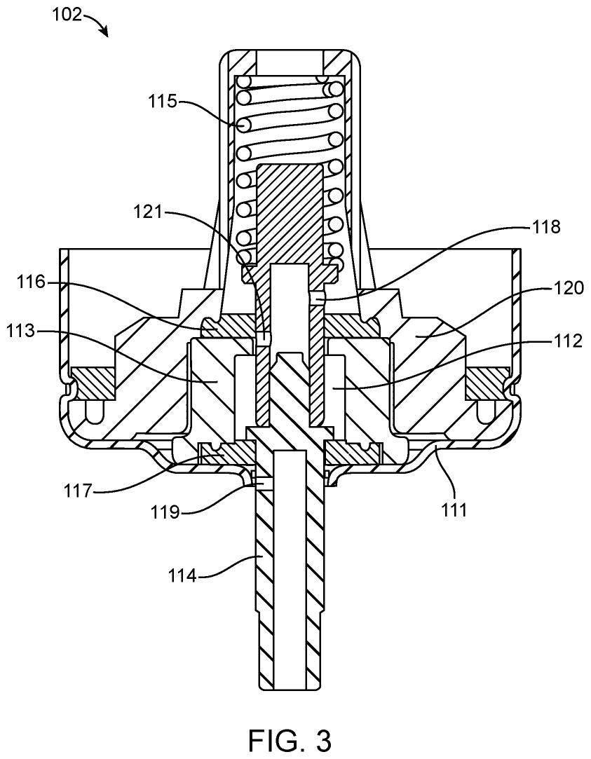

FIG. 3 shows another embodiment of a metered dose aerosol metering valve 102, different from the embodiment shown in FIGS. 1 and 2, in its rest position. The valve 102 has a metering chamber 112 defined in part by a metering tank 113 through which a stem 114 is biased outwardly by spring 115. The stem 114 is made in two parts that are push fit together before being assembled into the valve 102. The stem 114 has an inner seal 116 and an outer seal 117 disposed about it and forming sealing contact with the metering tank 113. A valve body 120 crimped into a ferrule 111 retains the aforementioned components in the valve. In use, formulation enters the metering chamber via orifices 121 and 118. The formulation's outward path from the metering chamber 112 when a dose is dispensed is via orifice 119.

### Propellant HFO-1234ze(E)

In certain embodiments, the primary propellant of compositions (i.e., formulations) according to the disclosure is HFO-1234ze(E), also known as trans-1,1,1,3-tetrafluoropropene, trans-1,3,3,3-tetrafluoropropene, or trans-1,3,3,3-tetrafluoroprop-1-ene. The chemical structure of the trans and cis isomers of HFO-1234ze are very different. As a result, these isomers have very different physical and thermodynamic properties. The significantly lower boiling point and higher vapor pressure of the trans (E) isomer relative to that of the cis (Z) isomer at ambient conditions makes the trans isomer a far more thermodynamically suitable propellant for achieving efficient pMDI atomization.

In some embodiments, the amount of HFO-1234ze(E) by weight in the formulation is at least 70%, greater than 70%, at least 80%, greater than 80%, at least 85%, greater than 85%, at least 90%, greater than 90%, at least 95%, or greater than 95%. In some embodiments, the amount of HFO-1234ze(E) in the formulation by weight is between 80% and 99%, between 80% and 98%, between 80% and 95%, or between 85% and 90%.

In some embodiments, the propellant is at least 90% or at least 95% of HFO-1234ze (E) with a minor amount of another propellant. In some of these embodiments, other propellants, such as hydrofluoroalkanes (e.g., HFA-134a, HFA-227, or HFA-152a) may be included as a minor component. Still other propellants that may be included as a minor component include other hydrofluroroolefins, including HFO-1234yf (C₃H₂F₄, 2,3,3,3-Tetrafluoroprop-1-ene) and HFO-1234ze (Z) (i.e., cis-HFO-1234ze). Amounts of such secondary propellants can include 0.1% to 10%, 0.5% to 5%, 1% to 5%, or 5% to 10% by weight, of the propellant. Thus, in some embodiments, the differences between HFA-152a and HFO-1234ze(E) discussed herein can be utilized to advantage by using a minor amount of HFA-152a. For example, in some embodiments, a minor amount of HFA-152a may be used to inhibit deposition of API particles on the surfaces of the metered dose inhaler that are contacted by the formulation as it passes from the canister in which it is stored to the nozzle outlet.

In some embodiments, the amount of HFO-1234ze(E) by weight of the total propellant in the formulation is at least 90%, at least 95%, at least 98%, at least 99%, at least 99.5%, or at least 99.8%. In some embodiments, HFO-1234ze(E) is the sole propellant in the composition. That is, the pharmaceutical product performance parameters, such as emitted dose and emitted particle size distribution, are not significantly different than if HFO-1234ze(E) were the sole propellant in the composition.

The propellant HFO-1234ze(E) is very different from the alternative low GWP propellant HFA-152a, as well as other propellants such as HFA-227 and HFA-134a. These propellants have different physical, chemical, and thermodynamic properties such as boiling point, vapor pressure, water solubility, liquid density, surface tension, etc. The differences in these properties make replacing one propellant with another without significantly compromising or altering pMDI product performance difficult to achieve. For example, the thermodynamic differences in propellant boiling point and vapor pressure can significantly affect pMDI aerosolization efficiency and give rise to differences in primary and secondary atomization mechanisms. Density differences between the liquid propellants and suspended API particles can affect suspension behavior, such as sedimentation rate. Differences in hygroscopicity between the propellants can affect moisture uptake, which could be problematic for suspension formulations, particularly if physical stability of the product (e.g., the suspended API particles) due to moisture uptake or chemical degradation in which water is involved is likely. Chemical interactions of the different propellants with APIs and excipients may also be significantly different, which could affect the long-term chemical stability of the product over the intended shelf life. Different propellants interact chemically and physically differently with valve plastics and elastomeric components, which could give rise to differences in the types and amounts of extractables and leachables, as well as impacting mechanical valve function or unit leakage. The thermodynamic properties of the propellants can give rise to different residual droplet/particle sizes due to differences in initial atomization and subsequent droplet evaporation rates and can also result in differences in spray characteristics such as spray force, temperature, velocity, and spray duration. Historically, the transition from CFC to HFA propellants has required significant efforts to develop new approaches to reformulate and develop capable hardware to achieve appropriate pMDI product performance. That is, it was not possible to simply directly substitute one propellant for another. Changing from a propellant such as HFA-152a to HFO-1234ze(E), or HFA-227 to HFO-1234ze(E), or HFA-134a to HFO-1234ze(E), for example, in a pMDI, is equally challenging due to many of the factors highlighted above.

In general, aerosols with mass median aerodynamic diameters (MMAD) of at least 1 μm but less than 5 μm in size are suitable for effective deep lung deposition for therapeutic effect. Herein, fine particle fraction (FPF) is defined as the percentage of the ex-actuator delivered API having a MMAD of less than 5 μm when tested in vitro. Herein, “ex-actuator” delivered dose is used to describe the amount of API delivered past the actuator of a pMDI. Therefore, an aerosol with a high FPF is typically suitable for deep lung deposition.

It was surprisingly found that pMDIs including APIs in suspension in HFO-1234ze (E) delivered a dose comprising a higher FPF than comparable pMDI suspensions in HFA-152a using comparable components when tested in vitro. In some embodiments, a metered dose inhaler including a suspension of at least one API in HFO-1234ze(E) delivers a dose comprising at least 15%, at least 17.5%, at least 20%, at least 25%, at least 30%, at least 35%, at least 40%, at least 45%, at least 50%, at least 55%, at least 60%, at least 65%, at least 70%, or at least 75% fine particle fraction of the at least one API.

### Propellant HFA-152a

In certain embodiments, the primary propellant of compositions (i.e., formulations) according to the disclosure is HFA-152a, also known as HFC-152a, R-152a, 1,1-difluoroethane, or DFE.

In some embodiments, the amount of HFA-152a by weight in the formulation is at least 70%, greater than 70%, at least 80%, greater than 80%, at least 85%, greater than 85%, at least 90%, greater than 90%, at least 95%, or greater than 95%. In some embodiments, the amount of HFA-152a in the formulation by weight is between 80% and 99%, between 80% and 98%, between 80% and 95%, or between 85% and 90%.

In some embodiments, the propellant is at least 90% or at least 95% of HFA-152a with a minor amount of another propellant. In some of these embodiments, other propellants, such as hydrofluoroalkanes (e.g., HFA-134a or HFA-227) may be included as a minor component. Still other propellants that may be included as a minor component include other hydrofluroroolefins, including HFO-1234yf, HFO-1234ze(E), and HFO-1234ze (Z) (i.e., cis-HFO-1234ze). Amounts of such secondary propellants can include 0.1% to 10%, 0.5% to 5%, 1% to 5%, or 5% to 10% by weight, of the composition (i.e., formulation). In some embodiments, the amount of HFA-152a by weight of the total propellant in the formulation is at least 90%, at least 95%, at least 98%, at least 99%, at least 99.5%, or at least 99.8%. In some embodiments, HFA-152a is the sole propellant in the composition. That is, the pharmaceutical product performance parameters, such as emitted dose and emitted particle size distribution, are not significantly different than if HFA-152a were the sole propellant in the composition.

The propellant HFA-152a is very different from the alternative low GWP propellant HFA-1234ze(E), as well as other propellants such as HFA-227 and HFA-134a. These propellants have different physical, chemical, and thermodynamic properties such as boiling point, vapor pressure, water solubility, liquid density, and surface tension. The differences in these properties make replacing one propellant with another, such as HFA-152a, without significantly compromising or altering pMDI product performance difficult to achieve. For example, the thermodynamic differences in propellant boiling point and vapor pressure can significantly affect pMDI aerosolization efficiency and give rise to differences in primary and secondary atomization mechanisms. Density differences between the liquid propellants and suspended API particles can affect suspension behavior, such as sedimentation rate. Differences in hygroscopicity between the propellants can affect moisture uptake, which could be problematic for suspension formulations, particularly if physical stability of the product (e.g., the suspended API particles) due to moisture uptake or chemical degradation in which water is involved is likely. Chemical interactions of the different propellants with APIs and excipients may also be significantly different, which could affect the long-term chemical stability of the product over the intended shelf life. Different propellants interact chemically and physically differently with valve plastics and elastomeric components, which could give rise to differences in the types and amounts of extractables and leachables, as well as impacting mechanical valve function or unit leakage. The thermodynamic properties of the propellants can give rise to different residual droplet/particle sizes due to differences in initial atomization and subsequent droplet evaporation rates and can also result in differences in spray characteristics such as spray force, temperature, velocity, and spray duration. Historically, the transition from CFC to HFA propellants has required significant efforts to develop new approaches to reformulate and develop capable hardware to achieve appropriate pMDI product performance. That is, it was not possible to simply directly substitute one propellant for another. Changing from a propellant such as HFO-1234ze(E), HFA-227, or HFA-134a to HFA-152a in a pMDI is equally challenging due to many of the factors highlighted above.

In some embodiments, a metered dose inhaler including a suspension of at least one API in HFA-152a delivers an ex-actuator dose comprising at least 10%, at least 15%, at least 17.5%, at least 20%, at least 25%, at least 30%, at least 35%, at least 40%, at least 45%, at least 50%, at least 55%, at least 60%, or at least 65% FPF of the at least one API.

### Active Pharmaceutical Ingredients

The API may be a drug (e.g., small-molecule drug), vaccine, DNA fragment, hormone, other treatment, or a combination of any two or more APIs. In certain embodiments, the formulations may include at least two (in certain embodiments, two or three, and in certain embodiments, two) APIs in suspension.

For preparation of suspension formulations, the API is preferably provided as a micronized crystalline solid. However, it should be apparent to one of ordinary skill in the art that other forms of API may be suitable for preparation of suspension formulations consistent with this disclosure. Examples include API prepared by micronization (e.g. ball or jet milling), spray drying, freeze drying, spray freeze drying, high-pressure homogenization, supercritical fluid technology, controlled crystallization, sonocrystallization, wet polishing, etc.

The API is in a pharmaceutically acceptable form. The API may be in free base form, a salt form (e.g., inorganic salts such as sodium salts, organic salts such as sulfate salts, esylate, etc.), or an ester form (e.g., propionate, or furoate), all of which are pharmaceutically acceptable. The API may be in a solvate form, hydrate form, or anhydrous form, all of which are pharmaceutically acceptable.

As used herein, the phrase “pharmaceutically acceptable” means that the component does not initiate a pharmacological response or an adverse reaction when introduced to a relevant biological system. By way of non-limiting example only, a substance found in the U.S. Food & Drug Administration's “Generally Recognized as Safe” (“GRAS”) list, or a substance used in accordance with guidelines in its Inactive Ingredient Database, would be considered pharmaceutically acceptable. Similarly, a substance in a corresponding database or list maintained by a parallel regulatory body, such as the European Medicines Agency, would be considered pharmaceutically acceptable. In general, in the formulations of the disclosure, it is desirable to employ only components that do not cause an unacceptable level of physical or chemical instability in the resulting composition.

Exemplary APIs can include those for the treatment of respiratory disorders, e.g., a bronchodilator, such as a short- or long-acting beta agonist, an anti-inflammatory (e.g., a corticosteroid), an anti-allergic, an anti-asthmatic, an antihistamine, a short-acting muscarinic antagonist (SAMA), a long-acting muscarinic antagonist (LAMA), a phosphodiesterase-4 (PDE4) inhibitor, a tyrosine kinase (TYK) inhibitor a Janus kinase (JAK) inhibitor, an antibiotic (e.g. aminoglycoside), an anti-infective, or an anticholinergic agent.

In certain embodiments, at least one API is a mast cell stabilizer, a receptor tyrosine kinase inhibitor, a beta-2 adrenergic receptor agonist, a steroid, or a combination thereof (e.g., a combination of one, two, three, or more different APIs).

Exemplary APIs can include salbutamol (i.e., albuterol), levalbuterol, terbutaline, ipratropium, oxitropium, tiotropium, beclomethasone, flunisolide, budesonide, mometasone, ciclesonide, cromolyn sodium, nedocromil sodium, ketotifen, azelastine, ergotamine, cyclosporine, aclidinium, umeclidinium, glycopyrronium (i.e., glycopyrrolate), salmeterol, fluticasone, formoterol, procaterol, indacaterol, carmoterol, milveterol, olodaterol, vilanterol, abediterol, omalizumab, zileuton, insulin, pentamidine, calcitonin, leuprolide, alpha-I-antitrypsin, interferon, triamcinolone, nintedanib, cromoglycate, a pharmaceutically acceptable salt or ester of any of the listed APIs, or a mixture of any of the listed APIs, their pharmaceutically acceptable salts or their pharmaceutically acceptable esters.

In certain embodiments, at least one API includes nintedanib or a pharmaceutically acceptable free base or salt thereof.

In certain embodiments, at least one API includes mometasone or a pharmaceutically acceptable free base or ester thereof. An example is mometasone furoate.

In certain embodiments, at least one API includes cromoglycate or a pharmaceutically acceptable salt thereof. An example is sodium cromoglycate (i.e., cromolyn sodium).

In certain embodiments, at least one API includes salbutamol (i.e., albuterol) or a pharmaceutically acceptable free base or salt thereof. An example is salbutamol sulfate (i.e., albuterol sulfate).

In all embodiments, the API(s) are dispersed or suspended in the formulation (i.e., as a suspension). In the event a combination of two or more APIs are used, all of the APIs are suspended. Where API is present in particulate form, i.e., suspended, it will generally have a mass median aerodynamic diameter in the range of 1 micrometer (μm) to 10 μm, preferably 1 μm to 5 μm.

### Formulations

The amount of API may be determined by the required dose per actuation and the pMDI metering valve size (i.e., volume), that is, the size of the metering chamber. In certain embodiments, the metering valve volume may be at most 100 microliters (μL or mcl) or at most 75 microliters. In certain embodiments, the metering valve volume may be at least 25 microliters.

The total amount of composition is desirably selected so that at least a portion of the propellant in the canister is present as a liquid after a predetermined number of medicinal doses have been delivered. The predetermined number of doses may be about 30 to about 200, about 60 to about 200, about 60 to about 120, about 60, about 120, about 200, or any other number of doses. The total amount of composition in the canister may be from about 1.0 to about 30.0 g, about 2.0 and about 20.0 g, about 5.0 and about 10.0 g. The total amount of composition is typically selected to be greater than the product of the predetermined number of doses times the metering volume of the metered valve. In some embodiments, the total amount of composition is greater than about 1.1, about 1.2, about 1.3, about 1.4, or about 1.5 times the product of the predetermined number of doses times the metering volume of the metered valve. This ensures that the amount of each dose remains relatively constant through the life of the inhaler.

In certain embodiments, the formulation includes at least 5 mg/mL, at least 10 mg/mL, at least 20 mg/mL, or at least 30 mg/mL of at least one API. In certain embodiments, the formulation includes at most 40 mg/mL of at least one API. In embodiments wherein there are at least two APIs included in the formulation, the formulation may include at least 0.1 mg/mL, at least 0.5 mg/mL, at least 1.0 mg/mL, at least 2.0 mg/mL, at least 5.0 mg/mL, at least 10 mg/mL, or at least 20 mg/mL of each API. In embodiments wherein there are at least two APIs included in the formulation, the formulation may include at most 35 mg/mL, at most 37.5 mg/mL, at most 39 mg/mL, or at most 39.9 mg/mL of each API. For example, a formulation may include 0.1 mg/mL to 39.9 mg/mL of a first API and 0.1 mg/mL to 39.9 mg/mL of a second API, such as 10 mg/mL of a first API and 30 mg/mL of a second API.

In certain embodiments, the formulation includes at least 0.5% by weight, at least 1% by weight, at least 2% by weight or at least 3% by weight of at least one API. In certain embodiments, the formulation includes at most 5% by weight of at least one API. In embodiments wherein a formulation includes more than one API, the formulation may include at least 0.5%, at least 1%, or at least 4% by weight of each API. In embodiments wherein a formulation includes more than one API, the formulation may include at most 4.5% by weight of each API. For example, a formulation may include 0.5% to 4.5% by weight of a first API and 0.5% to 4.5% by weight of a second API, such as 2% by weight of a first API and 3% by weight of a second API.

In certain embodiments, typical formulations of the present disclosure include the API or combination of APIs in an amount of at least 0.5 milligram per actuation (mg/actuation) (500 micrograms (μg, mcg) per actuation), or at least 1.0 mg/actuation (1000 μg/actuation). In certain embodiments, typical formulations of the present disclosure include the API in an amount of at most 2.0 mg/actuation (2000 μg/actuation).

It is known that high-dose pMDI suspensions can suffer from dose sampling inconsistencies due to poor homogeneity of the suspension. This can result from particle interactions with other particles or internal surfaces (e.g., deposition), and/or undesirable flocculation, agglomeration or phase separation of the high concentration of solid suspended particulates in the liquid formulation, which may be further exacerbated without the addition of excipients. These phenomena can lead to the non-ideal dispersion of suspended API in high dose pMDI suspension formulations causing inconsistent valve sampling of the formulation and reduced dose to dose consistency through unit life. Additionally, interaction of API particles with valve components and elastomers can affect valve functionality and/or lead to clogging of the valve or actuator orifice, also resulting in inconsistent or incomplete dose delivery.

Another consideration affecting high dose pMDI suspension performance is that the high concentration of suspended particles in the formulation leads to reduced aerosolization efficiency. In high API concentration suspensions, the number of particles contained within the resulting aerosol droplets will be higher than typical low-dose suspension pMDIs. Higher numbers of suspended particles in an atomized droplet of formulation retard the evaporation of that droplet and therefore the residual droplet size is often larger. Aerosols with larger residual droplet sizes have lower FPFs and are more likely to deposit in the oropharynx, reducing the therapeutic dose attainable in the lung.

In some embodiments, additional components, such as excipients and cosolvents beyond propellant and API can be added to the formulation. These components may have various uses and functions, including, but not limited to, facilitating formation of a suspension, stabilizing a suspension and aiding mechanical functionality of the unit.

Preferably, the suspension formulations of the present disclosure are substantially free of excipients (e.g., acids, surfactants), substantially free of cosolvents (e.g., alcohol, water), or substantially free of both excipients and cosolvents. In this context, “substantially free” means that excipients and cosolvents are not intentionally added to the formulation but may be present at trace levels. In addition, the product performance parameters, such as emitted dose and emitted particle size distribution, are not significantly different than a comparable formulation without excipients or cosolvents. In some embodiments, the amount of excipient or cosolvent by weight of the total composition is 2% or less, 1% or less, 0.5% or less, or 0.2% or less.

It has been previously demonstrated that for suspension formulations of an API at a concentration of less than 10 mg/mL in HFO-1234ze(E), inclusion of ethanol increased stability of the API in the suspension and reduced deposition of the API. Deposition and decreased stability of the API often result in API build-up and/or occlusion of the valve and/or actuator nozzle following multiple actuations. This build-up results in dose inconsistency. It would therefore be anticipated that a suspension formulation including an API at a concentration of 10 mg/mL or greater without an excipient or cosolvent (e.g., ethanol) would exhibit decreased stability and increased deposition of the API, and ultimately deliver an inconsistent dose.

Surprisingly, the data presented herein demonstrate that for a suspension formulation including one or more APIs in HFO-1234ze(E) or HFA-152a at a concentration of 10 mg/mL or greater, ethanol does not significantly change the stability or deposition of the one or more APIs. Therefore, pMDIs including suspension formulations without any excipient or cosolvent (e.g., ethanol) in either HFO-1234ze(E) or HFA-152a produce consistent through life dose delivery. Thus, suspensions of the present disclosure do not require ethanol.

While the suspension formulations of the present disclosure do not require a cosolvent such as ethanol, it may be desirable to include a small amount of ethanol for some applications. In some embodiments, a pMDI includes a suspension formulation including a low amount of ethanol by weight. For example, a formulation may include at most 5%, at most 4%, at most 3%, at most 2% or at most 1% of a cosolvent such as ethanol by weight. For example, a formulation may include from 0.5% to 5% of ethanol by weight, from 1% to 4% of ethanol by weight, or from 2% to 3% of ethanol by weight.

In certain embodiments, small amounts of water may be in suspension formulations. Preferably, however, added water is not used in making suspension formulations of the present disclosure.

### Metered Dose Inhalers

Returning to FIG. 1, in use, the patient actuates the inhaler 100 by pressing downwardly on the canister 1. This moves the canister 1 into the body of the actuator 5 and presses the valve stem 14 against the actuator stem socket 8 resulting in the canister metering valve 10 opening and releasing a metered dose of composition that passes through the actuator nozzle 7 and exits the mouthpiece 6 into the patient's mouth. It should be understood that other modes of actuation, such as breath-actuation, may be used as well and would operate as described with the exception that the force to depress the canister would be provided by the device, for instance by a spring or a motor-driven screw, in response to a triggering event, such as patient inhalation.

Devices that may be used with medicament formulations of the present disclosure include those described in U.S. Pat. No. 6,032,836 (Hiscocks et al.), U.S. Pat. No. 9,010,329 (Hansen), and U.K. Patent GB 2544128 B (Friel).

The metered dose inhaler can include a dose counter for counting the number of doses. Suitable dose counters are known in the art, and are described in, for example, U.S. Pat. No. 8,740,014 (Purkins et al.); U.S. Pat. No. 8,479,732 (Stuart et al.), and U.S. Pat. No. 8,814,035 (Stuart), and U.S. Patent Application Publication No. 2012/0234317 (Stuart), all of which are incorporated by reference in their entirety with respect to their disclosures of dose counters.

At least one of the various internal components of an inhaler, such as a metered dose inhaler, as described herein, such as one or more of the canister, valve, gaskets, seals, or O-rings, can be coated with one or more coatings. Some of these coatings provide a low surface energy. Such coatings are not always required because they are not always necessary for the successful operation of all inhalers. Thus, some metered dose inhalers do not include coated internal components.

Some coatings that can be used are described in U.S. Pat. No. 8,414,956 (Jinks et al.), U.S. Pat. No. 8,815,325 (David et al.), and U.S. Patent Application Publication No. 2012/0097159 (Iyer et al.), all of which are incorporated by reference in their entireties for their disclosure of coatings for inhalers and inhaler components. Other coatings, such as fluorinated ethylene propylene resins, or FEP, are also suitable. FEP is particularly suitable for use in coating canisters.

Some coating systems that can be used are described in European Patent Application No. 3661577 (Jinks et al.), European Patent No. 3146000 (Jinks et al.), and European Patent No. 3561004 (Jinks et al.). These coating systems are particularly useful for coating valves components, including one or more of valve stems, bottle emptiers, springs, and tanks. This coating system can be used with any type of inhaler and any formulation described herein.

In some embodiments, the actuator nozzle is sized so as to optimize the fine particle fraction and/or respirable dose delivered of the formulation within the canister when aerosolized. As described above, a high FPF is typically desirable for a pMDI. The FPF is determined by the formulation within a pMDI as well as by the physical components of the pMDI, such as the actuator nozzle. In some embodiments the actuator nozzle may have a nominal exit orifice diameter of at least 0.18 mm, such as at least 0.2 mm, at least 0.24 mm, 0.25 mm, at least 0.3 mm, or at least 0.35 mm. In some embodiments the nominal exit orifice diameter of the actuator nozzle may be 0.5 mm or less (i.e., at most 0.5 mm), such as 0.45 mm or less, 0.4 mm, 0.35 mm or less, 0.3 mm or less, or 0.25 mm or less. The actuator nozzle may have an exit orifice diameter of 0.3 mm to 0.5 mm, such as 0.35 mm to 0.45 mm, or 0.38 mm to 0.43 mm.

As used herein, the “nominal diameter” of the actuator nozzle exit orifice refers to a given diameter with slight variation due to manufacturing tolerances. For example, typical nozzle manufacturing allows for approximately +/−0.02 mm. Thus, a nominal diameter of 0.4 mm (or 0.40 mm) may be between 0.38 mm and 0.42 mm. In some embodiments, the cross-sectional shape of the actuator nozzle is essentially circular or circular and has a predetermined diameter. In some embodiments where the cross-sectional shape of the actuator nozzle is non-circular, for example oval, an effective diameter may be determined by taking an average over the distances spanning the opening (e.g., the average of major and minor axes of an ellipse).

It should be appreciated by one of ordinary skill in the art that a given actuator nozzle exit orifice may not be suitable for delivery of any formulation, and that selection of a suitable actuator nozzle exit orifice for a given formulation involves considerable effort.

In some embodiments, the MDI is manufactured by pressure filling. In pressure filling, the powdered medicament, optionally combined with one or more excipients (e.g., cosolvents), is placed in a suitable aerosol container (i.e., canister) capable of withstanding the vapor pressure of the propellant and fitted with a metering valve prior to filling. The propellant is then forced as a liquid through the valve into the container. In an alternate process of pressure filling, the particulate API is combined in a process vessel with propellant and optionally one or more excipients (e.g., cosolvents), and the resulting API suspension is transferred through the metering valve fitted to a suitable MDI container.

In some embodiments, the MDI is manufactured by cold filling. In cold filling, the powdered medicament, propellant which is chilled below its boiling point and, optionally, one or more excipients (e.g., cosolvents) are added to the MDI container. In addition, a metering valve is fitted to the container post filling.

For both pressure filling and cold filling processes, additional steps, such as mixing, sonication, and homogenization of the formulation may be optionally employed.

### EMBODIMENTS

### HFO-1234ze(E) Embodiments

Embodiment A1 is a metered dose inhaler comprising: a metering valve, a canister, and an actuator comprising an actuator nozzle, wherein the canister comprises a formulation, the formulation comprising greater than 70% by weight of HFO-1234ze(E), and at least one active pharmaceutical ingredient suspended in the formulation; wherein the metered dose inhaler delivers at least 0.5 milligram (mg) of the at least one active pharmaceutical ingredient per actuation.

Embodiment A2 is the metered dose inhaler of embodiment A1, wherein the formulation comprises at least 95% by weight of HFO-1234ze(E). Embodiment A3 is the metered dose inhaler of embodiment A2, wherein HFO-1234ze(E) is the sole propellant.

Embodiment A4 is the metered dose inhaler of any preceding A embodiment wherein the metered dose inhaler delivers a dose comprising at least 20% fine particle fraction of the at least one active pharmaceutical ingredient. Embodiment A5 is the metered dose inhaler of embodiment A4, wherein the metered dose inhaler delivers a dose comprising at least 30% fine particle fraction of the at least one active pharmaceutical ingredient. Embodiment A6 is the metered dose inhaler of embodiment A5, wherein the metered dose inhaler delivers a dose comprising at least 40% fine particle fraction of the at least one active pharmaceutical ingredient. Embodiment A7 is the metered dose inhaler of embodiment A6, wherein the metered dose inhaler delivers a dose comprising at least 60% fine particle fraction of the at least one active pharmaceutical ingredient.

Embodiment A8 is the metered dose inhaler of any preceding A embodiment, wherein the metered dose inhaler delivers at least 1.0 mg of the at least one active pharmaceutical ingredient per actuation. Embodiment A9 is the metered dose inhaler of any preceding A embodiment, wherein the metered dose inhaler delivers at most 2.0 mg of the at least one active pharmaceutical ingredient per actuation.

Embodiment A10 is the metered dose inhaler of any preceding A embodiment, wherein the at least one active pharmaceutical ingredient comprises a mast cell stabilizer, a receptor tyrosine kinase inhibitor, a beta-2 adrenergic receptor agonist, a steroid, or a combination thereof. Embodiment A11 is the metered dose inhaler of embodiment A10, wherein the at least one active pharmaceutical ingredient comprises nintedanib or a pharmaceutically acceptable free base or salt thereof. Embodiment A12 is the metered dose inhaler of embodiment A10, wherein the at least one active pharmaceutical ingredient comprises mometasone or a pharmaceutically acceptable ester or free base thereof. Embodiment A13 is the metered dose inhaler of embodiment A10, wherein the at least one active pharmaceutical ingredient comprises cromoglycate or a pharmaceutically acceptable salt thereof. Embodiment A14 is the metered dose inhaler of embodiment A10, wherein the at least one active pharmaceutical ingredient comprises salbutamol or a pharmaceutically acceptable salt or free base thereof.

Embodiment A15 is the metered dose inhaler of any preceding A embodiment, wherein the at least one active pharmaceutical ingredient is a micronized crystalline solid.

Embodiment A16 is the metered dose inhaler of any preceding A embodiment, wherein the formulation is substantially free of excipients. Embodiment A17 is the metered dose inhaler of any preceding A embodiment, wherein the formulation is substantially free of cosolvents. Embodiment A18 is the metered dose inhaler of any preceding A embodiment, wherein the formulation is substantially free of water. Embodiment A19 is the metered dose inhaler of any preceding A embodiments, wherein the formulation is substantially free of ethanol.

Embodiment A20 is the metered dose inhaler of any of embodiments A1 to A16, wherein the formulation comprises at most 5% of ethanol by weight. Embodiment A21 is the metered dose inhaler of embodiment A20, wherein the formulation comprises at most 2% of ethanol by weight.

Embodiment A22 is the metered dose inhaler of any preceding A embodiment, wherein the actuator nozzle comprises an exit orifice diameter of at most 0.5 mm. Embodiment A23 is the metered dose inhaler of embodiment A22, wherein the actuator nozzle comprises an exit orifice diameter of at most 0.25 mm. Embodiment A24 is the metered dose inhaler of any preceding A embodiment, wherein the actuator nozzle comprises an exit orifice diameter of at least 0.18 mm.

Embodiment A25 is the metered dose inhaler of any preceding A embodiment, wherein the metering valve has a volume of at most 100 microliters. Embodiment A26 is the metered dose inhaler of embodiment A25, wherein the metering valve has a volume of at most 75 microliters. Embodiment A27 is the metered dose inhaler of any preceding A embodiment, wherein the metering valve has a volume of at least 25 microliters.

Embodiment A28 is the metered dose inhaler of any preceding A embodiment, wherein the metering valve is coated or uncoated.

Embodiment A29 is the metered dose inhaler of any preceding A embodiment, wherein the metered dose inhaler comprises a coated or uncoated canister.

Embodiment A30 is the metered dose inhaler of any preceding A embodiment, wherein the formulation comprises at least 5 mg/mL of the at least one active pharmaceutical ingredient. Embodiment A31 is the metered dose inhaler of embodiment A30, wherein the formulation comprises at least 10 mg/mL of the at least one active pharmaceutical ingredient. Embodiment A32 is the metered dose inhaler of embodiment A31, wherein the formulation comprises at least 20 mg/mL of the at least one active pharmaceutical ingredient. Embodiment A33 is the metered dose inhaler of any preceding A embodiment, wherein the formulation comprises at most 40 mg/mL of the at least one active pharmaceutical ingredient.

Embodiment A34 is the metered dose inhaler of any preceding A embodiment, wherein the formulation comprises at least 0.5% by weight of the at least one active pharmaceutical ingredient. Embodiment A35 is the metered dose inhaler of embodiment A34, wherein the formulation comprises at least 1% by weight of the at least one active pharmaceutical ingredient. Embodiment A36 is the metered dose inhaler of embodiment A35, wherein the formulation comprises at least 2% by weight of the at least one active pharmaceutical ingredient. Embodiment A37 is the metered dose inhaler of embodiment A36, wherein the formulation comprises at least 3% by weight of the at least one active pharmaceutical ingredient. Embodiment A38 is the metered dose inhaler of any preceding A embodiment, wherein the formulation comprises at most 5% by weight of the at least one active pharmaceutical ingredient.

### HFA-152a Embodiments

Embodiment B1 is a metered dose inhaler comprising: a metering valve; a canister; and an actuator comprising an actuator nozzle; wherein the canister comprises a formulation, the formulation comprising greater than 70% by weight of HFA-152a; and at least one active pharmaceutical ingredient suspended in the formulation; wherein the metered dose inhaler delivers at least 0.5 milligram (mg) of the at least one active pharmaceutical ingredient per actuation.

Embodiment B2 is the metered dose inhaler of embodiment B1, wherein the formulation comprises at least 95% by weight of HFA-152a. Embodiment B3 is the metered dose inhaler of embodiment B2, wherein HFA-152a is the sole propellant.

Embodiment B4 is the metered dose inhaler of any preceding B embodiment, wherein the metered dose inhaler delivers at least 1.0 mg of the at least one active pharmaceutical ingredient per actuation. Embodiment B5 is the metered dose inhaler of any preceding B embodiment, wherein the metered dose inhaler delivers at most 2.0 mg of the at least one active pharmaceutical ingredient per actuation.

Embodiment B6 is the metered dose inhaler of any preceding B embodiment, wherein the at least one active pharmaceutical ingredient comprises a mast cell stabilizer, a receptor tyrosine kinase inhibitor, a beta-2 adrenergic receptor agonist, a steroid, or a combination thereof. Embodiment B7 is the metered dose inhaler of embodiment B6, wherein the at least one active pharmaceutical ingredient comprises nintedanib or a pharmaceutically acceptable salt or free base thereof. Embodiment B8 is the metered dose inhaler of embodiment B6, wherein the at least one active pharmaceutical ingredient comprises mometasone or a pharmaceutically acceptable ester or free base thereof.

Embodiment B9 is the metered dose inhaler of embodiment B6, wherein the at least one active pharmaceutical ingredient comprises cromoglycate or a pharmaceutically acceptable salt thereof. Embodiment B10 is the metered dose inhaler of embodiment B6, wherein the at least one active pharmaceutical ingredient comprises salbutamol or a pharmaceutically acceptable salt or free base thereof.

Embodiment B11 is the metered dose inhaler of any preceding B embodiment, wherein the at least one active pharmaceutical ingredient is a micronized crystalline solid.

Embodiment B12 is the metered dose inhaler of any preceding B embodiment, wherein the formulation is substantially free of excipients. Embodiment B13 is the metered dose inhaler of any preceding B embodiment, wherein the formulation is substantially free of cosolvents. Embodiment B14 is the metered dose inhaler of any preceding B embodiment, wherein the formulation is substantially free of ethanol. Embodiment B15 is the metered dose inhaler of any preceding B embodiment, wherein the formulation is substantially free of water.

Embodiment B16 is the metered dose inhaler of any of embodiments B1 to B12, wherein the formulation comprises at most 5% of ethanol by weight. Embodiment B17 is the metered dose inhaler of embodiment B16, wherein the formulation comprises at most 2% of ethanol by weight.

Embodiment B18 is the metered dose inhaler of any preceding B embodiment, wherein the actuator nozzle comprises an exit orifice diameter of at most 0.50 mm. Embodiment B19 is the metered dose inhaler of embodiment B18, wherein the actuator nozzle comprises an exit orifice diameter of at most 0.25 mm. Embodiment B20 is the metered dose inhaler of any preceding B embodiment, wherein the actuator nozzle comprises an exit orifice diameter of at least 0.18 mm.

Embodiment B21 is the metered dose inhaler of any preceding B embodiment, wherein the metered dose inhaler delivers a dose comprising at least 15% fine particle fraction of the at least one active pharmaceutical ingredient. Embodiment B22 is the metered dose inhaler of embodiment B21, wherein the metered dose inhaler delivers a dose comprising at least 30% fine particle fraction of the at least one active pharmaceutical ingredient. Embodiment B23 is the metered dose inhaler of embodiment B22, wherein the metered dose inhaler delivers a dose comprising at least 50% fine particle fraction of the at least one active pharmaceutical ingredient.

Embodiment B24 is the metered dose inhaler of any preceding B embodiment, wherein the metering valve has a volume of at most 100 microliters. Embodiment B25 is the metered dose inhaler of embodiment B24, wherein the metering valve has a volume of at most 75 microliters. Embodiment B26 is the metered dose inhaler of any preceding B embodiment, wherein the metering valve has a volume of at least 25 microliters.

Embodiment B27 is the metered dose inhaler of any preceding B embodiment, wherein metering valve is coated or uncoated.

Embodiment B28 is the metered dose inhaler of any preceding B embodiment, wherein the metered dose inhaler comprises a coated or uncoated canister. Embodiment B29 is the metered dose inhaler of any preceding B embodiment, wherein the formulation comprises at least 5 mg/mL of the at least one active pharmaceutical ingredient. Embodiment B30 is the metered dose inhaler of embodiment B29, wherein the formulation comprises at least 10 mg/mL of the at least one active pharmaceutical ingredient. Embodiment B31 is the metered dose inhaler of embodiment B30, wherein the formulation comprises at least 20 mg/mL of the at least one active pharmaceutical ingredient. Embodiment B32 is the metered dose inhaler of embodiment B31, wherein the formulation comprises at least 30 mg/mL of the at least one active pharmaceutical ingredient. Embodiment B33 is the metered dose inhaler of any preceding B embodiment, wherein the formulation comprises at most 40 mg/mL of the at least one active pharmaceutical ingredient.

Embodiment B34 is the metered dose inhaler of any preceding B embodiment, wherein the formulation comprises at least 0.5% by weight of the at least one active pharmaceutical ingredient. Embodiment B35 is the metered dose inhaler of embodiment B34, wherein the formulation comprises at least 1% by weight of the at least one active pharmaceutical ingredient. Embodiment B36 is the metered dose inhaler of embodiment B35, wherein the formulation comprises at least 2% by weight of the at least one active pharmaceutical ingredient. Embodiment B37 is the metered dose inhaler of embodiment B36, wherein the formulation comprises at least 3% by weight of the at least one active pharmaceutical ingredient. Embodiment B38 is the metered dose inhaler of any preceding B embodiment, wherein the formulation comprises at most 5% by weight of the at least one active pharmaceutical ingredient.

### EXAMPLES

In the following examples, dose consistency was determined by in vitro measurement of single actuation dose content at start, middle and end of pMDI unit life in line with U.S. Pharmacopeia (USP)<601>, using a flow rate of 28.3 L/min and apparatus A. Aerodynamic particle size distribution of the pMDI suspension aerosols was measured at start of unit life in line with U.S. Pharmacopeia (USP)<601> using a Next Generation impactor without pre-separator (apparatus 6) and a flow rate of 30 L/min.

### Example 1: Through Life Dose Consistency of Suspensions of Mometasone Furoate in HFA-152a or HFO-1234ze(E) with and without Ethanol

Suspensions of micronized mometasone furoate in HFA-152a or HFO-1234ze(E) were prepared. A first set of suspensions included no ethanol. A second set of suspensions included 2% ethanol by weight. Each suspension included an amount of mometasone furoate to provide a nominal delivered dose (ex-valve) of 0.5 mg/actuation (10.0 mg/mL) or 1.0 mg/actuation (20.0 mg/mL). Two further suspensions including no ethanol were prepared which included an amount of mometasone furoate to provide a nominal delivered dose (ex-valve) of 2.0 mg/actuation (40.0 mg/mL).

In total, ten suspension pMDI formulations were prepared in triplicate with sufficient fill weight to provide 60 actuations. Each suspension was filled into Kindeva FEP coated canisters, crimped with a Kindeva 50-μL valve. Each pMDI unit was sonicated for at least 10 minutes to disperse the suspended drug and tested with a Kindeva actuator having an exit orifice diameter (EOD) of 0.4 mm.

The through-life (TL) dose consistency of each pMDI unit was determined by measuring the mean ex-actuator delivered dose in micrograms of mometasone furoate (N=3) at the start of life (SoL), middle of life (MOL) and end of life (EoL) of the unit. These data are presented in Table 1.

TABLE 1
Mean delivered dose in micrograms of mometasone furoate (N = 3) at the start, middle
and end of unit life, through life mean and standard deviation of through life mean.

| | | | | | | TL mean |
|---|---|---|---|---|---|---|
| Nominal | | SoL | MoL | EoL | TL | delivered |
| ex-valve | | mean | mean | mean | mean | dose |
| delivered | | delivered | delivered | delivered | delivered | standard |
| dose | | dose | dose | dose | dose | deviation |
| (mg/act) | Propellant/ethanol | (μg/act) | (μg/act) | (μg/act) | (μg/act) | (μg/act) |
| 0.5 | HFA-152a | 457.7 | 433.8 | 433.2 | 441.6 | 42.5 |
| 0.5 | HFA-152a/2% EtOH (w/w) | 408.9 | 427.3 | 450.9 | 429.1 | 80.4 |
| 1.0 | HFA-152a | 1087.9 | 1018.3 | 705.6 | 937.3 | 197.4 |
| 1.0 | HFA-152a/2% EtOH (w/w) | 906.5 | 936.3 | 759.7 | 867.5 | 88.9 |
| 2.0 | HFA-152a | 1815.1 | 1771.5 | 1613.8 | 1733.4 | 159.6 |
| 0.5 | HFO-1234ze(E) | 457.5 | 434.7 | 430.8 | 441.0 | 32.4 |
| 0.5 | HFO-1234ze(E)/2% EtOH (w/w) | 433.9 | 453.1 | 465.7 | 450.9 | 67.8 |
| 1.0 | HFO-1234ze(E) | 752.4 | 931.9 | 934.4 | 855.3 | 466.2 |
| 1.0 | HFO-1234ze(E)/2% EtOH (w/w) | 852.6 | 1003.4 | 848.1 | 901.4 | 111.0 |
| 2.0 | HFO-1234ze(E) | 1755.0 | 1725.9 | 1837.0 | 1772.6 | 244.5 |

From this example, it was learned that pMDI suspensions of mometasone furoate in HFA-152a or HFO-1234ze(E) with and without 2% ethanol by weight produced relatively consistent through unit life ex-actuator delivered dose. Surprisingly, it was learned that the addition of ethanol did not always substantially improve through unit life ex-actuator delivered dose consistency.

### Example 2: Through Life Dose Consistency of Suspensions of Sodium Cromoglycate in HFA-152a or HFO-1234ze(E) with and without Ethanol

Suspensions of micronized sodium cromoglycate in HFA-152a or HFO-1234ze(E) were prepared. A first set of suspensions included no ethanol. A second set of suspensions included 2% ethanol by weight. Each suspension included an amount of sodium cromoglycate to provide a nominal delivered dose (ex-valve) of 0.5 mg/actuation (10.0 mg/mL) or 1.0 mg/actuation (20.0 mg/mL). Two further suspensions including no ethanol were prepared which included an amount of sodium cromoglycate to provide a nominal delivered dose (ex-valve) of 2.0 mg/actuation (40.0 mg/mL).

In total, ten suspension pMDI formulations were prepared in triplicate with sufficient fill weight to provide 60 actuations. Each suspension was filled into Kindeva FEP coated canisters, crimped with a Kindeva 50-μL valve. Each pMDI unit was sonicated for at least 10 minutes to disperse the suspended drug and tested with a Kindeva actuator having an exit orifice diameter (EOD) of 0.4 mm.

The through-life (TL) dose consistency of each pMDI unit was determined by measuring the mean ex-actuator delivered dose in micrograms of sodium cromoglycate (N=3) at the start of life (SoL), middle of life (MOL) and end of life (EoL) of the unit. These data are presented in Table 2.

TABLE 2
Mean delivered dose in micrograms of sodium cromoglycate (N = 3) at the start, middle
and end of unit life, through life mean and standard deviation of through life mean.

| | | | | | | TL mean |
|---|---|---|---|---|---|---|
| Nominal | | SoL | MoL | EoL | TL | delivered |
| ex-valve | | mean | mean | mean | mean | dose |
| delivered | | delivered | delivered | delivered | delivered | standard |
| dose | | dose | dose | dose | dose | deviation |
| (mg/act) | Propellant/ethanol | (μg/act) | (μg/act) | (μg/act) | (μg/act) | (μg/act) |
| 0.5 | HFA-152a | 611.8 | 363.8 | 307.9 | 427.8 | 159.1 |
| 0.5 | HFA-152a/2% EtOH (w/w) | 675.8 | 331.6 | 313.6 | 440.3 | 196.2 |
| 1.0 | HFA-152a | 950.3 | 931.3 | 618.4 | 833.3 | 175.3 |
| 1.0 | HFA-152a/2% EtOH (w/w) | 1027.9 | 906.3 | 847.0 | 927.0 | 173.4 |
| 2.0 | HFA-152a | 1782.4 | 1590.1 | 1694.4 | 1689.0 | 194.8 |
| 0.5 | HFO-1234ze(E) | 419.4 | 354.1 | 158.7 | 310.7 | 120.5 |
| 0.5 | HFO-1234ze(E)/2% EtOH (w/w) | 940.2 | 332.8 | 315.4 | 529.5 | 405.8 |
| 1.0 | HFO-1234ze(E) | 967.3 | 784.8 | 695.5 | 815.9 | 163.7 |
| 1.0 | HFO-1234ze(E)/2% EtOH (w/w) | 876.4 | 917.0 | 735.1 | 842.9 | 107.0 |
| 2.0 | HFO-1234ze(E) | 2009.3 | 879.1 | 119.8 | 1255.0 | 1209.9 |

From this example, it was learned that pMDI suspensions of sodium cromoglycate in HFA-152a or HFO-1234ze(E) with and without 2% ethanol by weight produced relatively consistent through unit life ex-actuator delivered dose. Surprisingly, it was learned that the addition of ethanol did not always substantially improve through unit life ex-actuator delivered dose consistency.

### Example 3: Through Life Dose Consistency of Suspensions of Salbutamol Sulfate in HFA-152a or HFO-1234ze(E)

Suspensions of micronized salbutamol sulfate in HFA-152a or HFO-1234ze(E) were prepared. Each suspension included an amount of salbutamol sulfate to provide a nominal delivered dose (ex-valve) of 0.5 mg/actuation (10.0 mg/mL), 1.0 mg/actuation (20.0 mg/mL) or 2.0 mg/actuation (40.0 mg/mL) and included no ethanol.

In total, six suspension pMDI formulations were prepared in triplicate with sufficient fill weight to provide 60 actuations. Each suspension was filled into Kindeva FEP coated canisters, crimped with a Kindeva 50-μL valve. Each pMDI unit was sonicated for at least 10 minutes to disperse the suspended drug and tested with a Kindeva actuator having an exit orifice diameter (EOD) of 0.4 mm.

The through-life (TL) dose consistency of each pMDI unit was determined by measuring the mean ex-actuator delivered dose in micrograms of salbutamol sulfate (N=3) at the start of life (SoL), middle of life (MOL) and end of life (EoL) of the unit. These data are presented in Table 3.

TABLE 3
Mean delivered dose in micrograms of salbutamol sulfate (N = 3) at the start, middle
and end of unit life, through life mean and standard deviation of through life mean.

| | | | | | | TL mean |
|---|---|---|---|---|---|---|
| Nominal | | SoL | MoL | EoL | TL | delivered |
| ex-valve | | mean | mean | mean | mean | dose |
| delivered | | delivered | delivered | delivered | delivered | standard |
| dose | | dose | dose | dose | dose | deviation |
| (mg/act) | Propellant | (μg/act) | (μg/act) | (μg/act) | (μg/act) | (μg/act) |
| 0.5 | HFA-152a | 310.1 | 307.7 | 308.5 | 308.8 | 31.5 |
| 1.0 | HFA-152a | 637.8 | 634.3 | 664.3 | 645.5 | 70.3 |
| 2.0 | HFA-152a | 1137.8 | 1097.0 | 1631.7 | 1190.9 | 216.9 |
| 0.5 | HFO-1234ze(E) | 309.9 | 289.8 | 288.5 | 296.1 | 53.8 |
| 1.0 | HFO-1234ze(E) | 477.0 | 584.2 | 705.1 | 588.8 | 135.2 |
| 2.0 | HFO-1234ze(E) | 1097.1 | 1232.6 | 1398.8 | 1223.3 | 189.9 |

From this example, it was learned that pMDI suspensions of salbutamol sulfate in HFA-152a or HFO-1234ze(E) produced relatively consistent through unit life ex-actuator delivered dose.

### Example 4: Through Life Dose Consistency of Suspensions of Nintedanib (Free Base) in HFA-152a or HFO-1234ze(E) with and without Ethanol

Suspensions of micronized nintedanib (free base) in HFA-152a or HFO-1234ze(E) were prepared. A first set of suspensions included no ethanol. A second set of suspensions included 2% ethanol by weight. Each suspension included an amount of nintedanib (free base) to provide a nominal delivered dose (ex-valve) of 0.5 mg/actuation (7.94 mg/mL) or 1.0 mg/actuation (15.87 mg/mL). Two further suspensions including no ethanol were prepared which included an amount of nintedanib (free base) to provide a nominal delivered dose (ex-valve) of 2.0 mg/actuation (20.0 mg/mL).

In total, ten suspension pMDI formulations were prepared in triplicate with sufficient fill weight to provide 60 actuations. The pMDI formulations containing 0.5 mg/actuation (7.94 mg/mL) and 1.0 mg/actuation (15.87 mg/mL) of nintedanib (free base) were filled into Kindeva FEP coated canisters, crimped with a Kindeva 63-μL valve. The pMDI formulations containing 2.0 mg/actuation (20.0 mg/mL) of nintedanib (free base) were filled into Kindeva FEP coated canisters, crimped with a Kindeva 100-μL valve. Each pMDI unit was sonicated for at least 10 minutes to disperse the suspended drug and tested with a Kindeva actuator having an exit orifice diameter (EOD) of 0.4 mm.

The through-life (TL) dose consistency of each pMDI unit was determined by measuring the mean ex-actuator delivered dose in micrograms of nintedanib (free base) (N=3) at the start of life (SoL), middle of life (MOL) and end of life (EoL) of the unit. These data are presented in Table 4.

TABLE 4
Mean delivered dose in micrograms of nintedanib (free base) (N = 3) at the start,
middle and end of unit life, through life mean and standard deviation of through life mean.

| Nominal | | SoL | MoL | EoL | TL | TL mean |
|---|---|---|---|---|---|---|
| ex-valve | | mean | mean | mean | mean | delivered |
| delivered | | delivered | delivered | delivered | delivered | dose |
| dose | | dose | dose | dose | dose | standard |
| (mg/act) | Propellant/ethanol | (μg/act) | (μg/act) | (μg/act) | (μg/act) | deviation |
| 0.5 | HFA152a | 468.9 | 360.0 | 335.9 | 388.3 | 68.7 |
| 0.5 | HFA152a/2% EtOH (w/w) | 452.4 | 364.6 | 383.1 | 400.1 | 43.4 |
| 1.0 | HFA152a | 1020.0 | 759.1 | 890.2 | 889.8 | 259.0 |
| 1.0 | HFA152a/2% EtOH (w/w) | 940.6 | 773.1 | 818.8 | 844.2 | 97.5 |
| 2.0 | HFA152a | 2205.7 | 1876.0 | 1954.6 | 2012.2 | 203.5 |
| 0.5 | HFO1234ze(E) | 401.8 | 372.8 | 380.5 | 385.0 | 24.2 |
| 0.5 | HFO1234ze(E)/2% EtOH (w/w) | 431.1 | 385.9 | 378.0 | 398.3 | 27.1 |
| 1.0 | HFO1234ze(E) | 947.8 | 962.2 | 947.3 | 952.4 | 86.2 |
| 1.0 | HFO1234ze(E)/2% EtOH (w/w) | 926.7 | 824.9 | 818.5 | 856.7 | 62.1 |
| 2.0 | HFO1234ze(E) | 1561.8 | 1884.0 | 2282.4 | 1909.4 | 611.1 |

From this example, it was learned that pMDI suspensions of nintedanib (free base) in HFA-152a or HFO-1234ze(E) with and without 2% ethanol by weight produced relatively consistent through unit life ex-actuator delivered dose. Surprisingly, it was learned that the addition of ethanol did not always substantially improve through unit life ex-actuator delivered dose consistency.

### Example 5: Aerodynamic Particle Size Measurement of Suspensions of Mometasone Furoate in HFA-152a or HFO-1234ze(E) with and without Ethanol

Suspensions of micronized mometasone furoate in HFA-152a or HFO-1234ze(E) were prepared. A first set of suspensions included no ethanol. A second set of suspensions included 2% ethanol by weight. Each suspension included an amount of mometasone furoate to provide a nominal delivered dose (ex-valve) of 0.5 mg/actuation (10.0 mg/mL) or 1.0 mg/actuation (20.0 mg/mL). Two further suspensions including no ethanol were prepared which included an amount of mometasone furoate to provide a nominal delivered dose (ex-valve) of 2.0 mg/actuation (40.0 mg/mL).

In total, ten suspension pMDI formulations were prepared in triplicate with sufficient fill weight to provide 60 actuations. Each suspension was filled into Kindeva FEP coated canisters, crimped with a Kindeva 50-μL valve. Each pMDI unit was sonicated for at least 10 minutes to disperse the suspended drug and tested with a Kindeva actuator having an exit orifice diameter (EOD) of 0.4 mm or 0.25 mm.

The mass median aerodynamic diameter (MMAD) and fine particle fraction (FPF) of each pMDI unit were measured by Next Generation cascade impaction (N=3) for each pMDI suspension. Results are shown in Table 5.

TABLE 5
Mean mass median aerodynamic diameter (MMAD)
and mean fine particle fraction (FPF) of
mometasone furoate (N = 3) aerosols

| | | Actuator exit orifice diameter | | | |
|---|---|---|---|---|---|
| Mometasone Furoate | | 0.4 mm | | 0.25 mm | |
| Nominal ex-valve | | Mean | Mean | Mean | Mean |
| delivered dose | | MMAD | FPF | MMAD | FPF |
| (mg/act) | Propellant/ethanol | (μm) | (%) | (μm) | (%) |
| 0.5 | HFA-152a | 3.56 | 37.5 | — | — |
| 0.5 | HFA-152a/2% | 3.55 | 34.0 | — | — |
| | EtOH (w/w) | | | | |
| 1.0 | HFA-152a | 4.13 | 29.4 | 3.75 | 49.1 |
| 1.0 | HFA-152a/2% | 3.90 | 31.1 | 3.91 | 45.3 |
| | EtOH (w/w) | | | | |
| 2.0 | HFA-152a | 5.09 | 19.5 | — | — |
| 0.5 | HFO-1234ze(E) | 3.36 | 49.8 | 3.41 | 66.6 |
| 0.5 | HFO-1234ze(E)/2% | 3.70 | 39.0 | 3.70 | 55.3 |
| | EtOH (w/w) | | | | |
| 1.0 | HFO-1234ze(E) | 3.69 | 39.5 | 3.61 | 58.8 |
| 1.0 | HFO-1234ze(E)/2% | 4.01 | 31.8 | 3.84 | 52.5 |
| | EtOH (w/w) | | | | |
| 2.0 | HFO-1234ze(E) | 4.87 | 22.5 | — | — |

It was observed that pMDI suspensions of mometasone furoate in HFA-152a or HFO-1234ze(E), with and without ethanol, produced aerosols with MMAD values in an appropriate respirable range required for therapeutic effect when tested in-vitro.

It was learned that pMDI suspensions of mometasone furoate in HFA-152a or HFO-1234ze(E) without ethanol generally produced greater FPFs than the corresponding pMDI suspensions of mometasone furoate with 2% ethanol by weight, for all nominal doses.

Surprisingly, pMDI suspensions of mometasone furoate in HFO-1234ze(E) consistently produced greater FPFs and generally smaller MMADs than the corresponding pMDI suspensions of mometasone furoate in HFA-152a, with or without ethanol. From this example it was learned that the aerosolization efficiency of pMDI suspensions of mometasone furoate in HFO-1234ze(E) was consistently better than when in HFA-152a.

It was learned that pMDI suspensions of 0.5 mg/actuation and 1.0 mg/actuation of micronized mometasone furoate in HFO-1234ze(E) and 1.0 mg/actuation pMDI suspensions of mometasone furoate in HFA-152a tested with a Kindeva actuator having an EOD of 0.25 mm produced consistently greater FPFs than with an actuator having an EOD of 0.4 mm and therefore decreasing the size of the actuator EOD significantly improved aerosolization efficiency.

### Example 6: Aerodynamic Particle Size Measurement of Suspensions of Sodium Cromoglycate in HFA-152a or HFO-1234ze(E) with and without Ethanol

Suspensions of micronized sodium cromoglycate in HFA-152a or HFO-1234ze(E) were prepared. A first set of suspensions included no ethanol. A second set of suspensions included 2% ethanol by weight. Each suspension included an amount of sodium cromoglycate to provide a nominal delivered dose (ex-valve) of 0.5 mg/actuation (10.0 mg/mL) or 1.0 mg/actuation (20.0 mg/mL). Two further suspensions including no ethanol were prepared which included an amount of sodium cromoglycate to provide a nominal delivered dose (ex-valve) of 2.0 mg/actuation (40.0 mg/mL).

In total, ten suspension pMDI formulations were prepared in triplicate with sufficient fill weight to provide 60 actuations. Each suspension was filled into Kindeva FEP coated canisters, crimped with a Kindeva 50-μL valve. Each pMDI unit was sonicated for at least 10 minutes to disperse the suspended drug and tested with a Kindeva actuator having an exit orifice diameter (EOD) of 0.4 mm

The mass median aerodynamic diameter (MMAD) and fine particle fraction (FPF) of each pMDI unit were measured by Next Generation cascade impaction (N=3) for each pMDI suspension. Results are shown in Table 6.

TABLE 6
Mean mass median aerodynamic diameter (MMAD)
and mean fine particle fraction (FPF) of
sodium cromoglycate (N = 3) aerosols

| Sodium Cromoglycate | | | |
|---|---|---|---|
| Nominal ex-valve | | Mean | Mean |
| delivered dose | | MMAD | FPF |
| (mg/act) | Propellant/ethanol | (μm) | (%) |
| 0.5 | HFA-152a | 3.59 | 33.0 |
| 0.5 | HFA-152a/2% EtOH (w/w) | 3.92 | 29.5 |
| 1.0 | HFA-152a | 3.85 | 28.2 |
| 1.0 | HFA-152a/2% EtOH (w/w) | 4.38 | 25.2 |
| 2.0 | HFA-152a | 4.39 | 24.7 |
| 0.5 | HFO-1234ze(E) | 3.41 | 41.3 |
| 0.5 | HFO-1234ze(E)/2% EtOH (w/w) | 3.77 | 34.1 |
| 1.0 | HFO-1234ze(E) | 3.72 | 34.7 |
| 1.0 | HFO-1234ze(E)/2% EtOH (w/w) | 4.13 | 30.9 |
| 2.0 | HFO-1234ze(E) | 4.23 | 30.7 |

It was observed that pMDI suspensions of sodium cromoglycate in HFA-152a or HFO-1234ze(E), with and without ethanol, produced aerosols with MMAD values in an appropriate respirable range required for therapeutic effect when tested in-vitro.

It was learned that pMDI suspensions of sodium cromoglycate in HFA-152a or HFO-1234ze(E) without ethanol consistently produced greater FPFs and smaller MMADs than the corresponding pMDI suspensions of sodium cromoglycate with 2% ethanol by weight, for all nominal doses.

Surprisingly, pMDI suspensions of sodium cromoglycate in HFO-1234ze(E) consistently produced greater FPFs and generally smaller MMADs than the corresponding pMDI suspensions of sodium cromoglycate in HFA-152a, with or without ethanol. From this example it was learned that the aerosolization efficiency of pMDI suspensions of sodium cromoglycate in HFO-1234ze(E) was consistently better than when in HFA-152a.

### Example 7: Aerodynamic Particle Size Measurement of Suspensions of Salbutamol Sulfate in HFA-152a or HFO-1234ze(E)

Suspensions of micronized salbutamol sulfate in HFA-152a or HFO-1234ze(E) were prepared. Each suspension included an amount of salbutamol sulfate to provide a nominal delivered dose (ex-valve) of 0.5 mg/actuation (10.0 mg/mL), 1.0 mg/actuation (20.0 mg/mL) or 2.0 mg/actuation (40.0 mg/mL) and included no ethanol.

In total, six suspension pMDI formulations were prepared in triplicate with sufficient fill weight to provide 60 actuations. Each suspension was filled into Kindeva FEP coated canisters, crimped with a Kindeva 50-μL valve. Each pMDI unit was sonicated for at least 10 minutes to disperse the suspended drug and tested with a Kindeva actuator having an exit orifice diameter (EOD) of 0.4 mm.

The mass median aerodynamic diameter (MMAD) and fine particle fraction (FPF) of each pMDI unit were measured by Next Generation cascade impaction (N=3) for each pMDI suspension. Results are shown in Table 7.

TABLE 7
Mean mass median aerodynamic diameter (MMAD)
and mean fine particle fraction (FPF) of
Salbutamol sulfate (N = 3) aerosols

| Salbutamol sulfate | | | |
|---|---|---|---|
| Nominal ex-valve | | Mean | Mean |
| delivered dose | | MMAD | FPF |
| (mg/act) | Propellant | (μm) | (%) |
| 0.5 | HFA-152a | 2.98 | 45.6 |
| 1.0 | HFA-152a | 3.30 | 40.8 |
| 2.0 | HFA-152a | 3.92 | 30.3 |
| 0.5 | HFO-1234ze(E) | 2.98 | 57.0 |
| 1.0 | HFO-1234ze(E) | 3.12 | 52.4 |
| 2.0 | HFO-1234ze(E) | 3.67 | 36.4 |

It was observed that pMDI suspensions of salbutamol sulfate in HFA-152a or HFO-1234ze(E) produced aerosols with MMAD values in an appropriate respirable range required for therapeutic effect when tested in-vitro.

Surprisingly, pMDI suspensions of salbutamol sulfate in HFO-1234ze(E) consistently produced greater FPFs and smaller MMADs than the corresponding pMDI suspensions of salbutamol sulfate in HFA-152a. From this example it was learned that the aerosolization efficiency of pMDI suspensions of salbutamol sulfate in HFO-1234ze(E) was consistently better than when in HFA-152a.

### Example 8: Aerodynamic Particle Size Measurement of Suspensions of Nintedanib (Free Base) in HFA-152a or HFO-1234ze(E) with and without Ethanol

Suspensions of micronized nintedanib (free base) in HFA-152a or HFO-1234ze(E) were prepared. A first set of suspensions included no ethanol. A second set of suspensions included 2% ethanol by weight. Each suspension included an amount of nintedanib (free base) to provide a nominal delivered dose (ex-valve) of 0.5 mg/actuation (7.94 mg/mL) or 1.0 mg/actuation (15.87 mg/mL). Two further suspensions including no ethanol were prepared which included an amount of nintedanib (free base) to provide a nominal delivered dose (ex-valve) of 2.0 mg/actuation (20.0 mg/mL).

In total, ten suspension pMDI formulations were prepared in triplicate with sufficient fill weight to provide 60 actuations. The pMDI formulations containing 0.5 mg/actuation (7.94 mg/mL) and 1.0 mg/actuation (15.87 mg/mL) of nintedanib (free base) were filled into Kindeva FEP coated canisters, crimped with a Kindeva 63-μL valve. The pMDI formulations containing 2.0 mg/actuation (20.0 mg/mL) of nintedanib (free base) were filled into Kindeva FEP coated canisters, crimped with a Kindeva 100-μL valve. Each pMDI unit was sonicated for at least 10 minutes to disperse the suspended drug and tested with a Kindeva actuator having an exit orifice diameter (EOD) of 0.4 mm. The mass median aerodynamic diameter (MMAD) and fine particle fraction (FPF) of each pMDI unit were measured by Next Generation cascade impaction (N=3) for each pMDI suspension. Results are shown in Table 8.

TABLE 8
Mean mass median aerodynamic diameter (MMAD)
and mean fine particle fraction (FPF) of nintedanib
(free base) (N = 3) aerosols

| Nintedanib (free base) | | | |
|---|---|---|---|
| Nominal ex-valve | | Mean | Mean |
| delivered dose | | MMAD | FPF |
| (mg/act) | Propellant/ethanol | (μm) | (%) |
| 0.5 | HFA-152a | 3.74 | 33.7 |
| 0.5 | HFA-152a/2% EtOH (w/w) | 3.65 | 26.9 |
| 1.0 | HFA-152a | 5.01 | 23.8 |
| 1.0 | HFA-152a/2% EtOH (w/w) | 4.74 | 18.0 |
| 2.0 | HFA-152a | 7.47 | 16.7 |
| 0.5 | HFO-1234ze(E) | 2.88 | 60.0 |
| 0.5 | HFO-1234ze(E)/2% EtOH (w/w) | 3.50 | 36.8 |
| 1.0 | HFO-1234ze(E) | 3.66 | 44.4 |
| 1.0 | HFO-1234ze(E)/2% EtOH (w/w) | 4.20 | 29.3 |
| 2.0 | HFO-1234ze(E) | 4.08 | 34.8 |

It was observed that pMDI suspensions of nintedanib (free base) in HFA-152a or HFO-1234ze(E), with and without ethanol, produced aerosols with MMAD values in an appropriate respirable range required for therapeutic effect when tested in-vitro.

It was learned that pMDI suspensions of nintedanib (free base) in HFA-152a or HFO-1234ze(E) without ethanol consistently produced greater FPFs and smaller MMADs than the corresponding pMDI suspensions of nintedanib (free base) with 2% ethanol by weight, for all nominal doses.

Surprisingly, pMDI suspensions of nintedanib (free base) in HFO-1234ze(E) consistently produced greater FPFs and smaller MMADs than the corresponding pMDI suspensions of nintedanib (free base) in HFA-152a, with or without ethanol. From this example it was learned that the aerosolization efficiency of pMDI suspensions of nintedanib (free base) in HFO-1234ze(E) was consistently better than when in HFA-152a.

### Example 9: Additional Through Life Dose Consistency of a Suspension of Nintedanib (Free Base) in HFO-1234ze(E) without Ethanol

A suspension of nintedanib was prepared in HFO-1234ze(E). The concentration of nintedanib (15.9 mg/mL) was selected to provide a nominal actuation dose of 1 mg/actuation from a 63-microliter valve. The bottle emptier, tank, spring, and ferrule components of the valves were coated with a fluoropolymer coating according to the general process described in Example 2 of U.S. Patent Application Publication 2017/0152396 A1 (Jinks et al., hereby incorporated by reference). The formulation was mixed with a high shear mixer and cold filled into 16 mL FEP-coated canisters.

Through life dosing content uniformity showed no trending between start, middle, and end of life with an average dose of 950 μg/actuation. Average fine particle mass (<5 μm) was 434 μg/actuation.

The embodiments described above and illustrated in the figures are presented by way of example only and are not intended as a limitation upon the concepts and principles of the present disclosure. As such, it will be appreciated by one having ordinary skill in the art that various changes in the elements and their configuration and arrangement are possible without departing from the spirit and scope of the present disclosure. All references and publications cited herein are expressly incorporated herein by reference in their entirety into this disclosure. Various features and aspects of the present disclosure are set forth in the following claims.

1. A metered dose inhaler comprising:
a metering valve;
a canister; and
an actuator comprising an actuator nozzle;
wherein the canister comprises a formulation, the formulation comprising greater than 70% by weight of HFO-1234ze(E); and
at least one active pharmaceutical ingredient suspended in the formulation;
wherein the metered dose inhaler delivers at least 0.5 milligram (mg) of the at least one active pharmaceutical ingredient per actuation.

2. The metered dose inhaler of claim 1, wherein the formulation comprises at least 95% by weight of HFO-1234ze(E).

3. The metered dose inhaler of claim 1, wherein the metered dose inhaler delivers an ex-actuator dose comprising at least 20% fine particle fraction of less than 5 μm in diameter of the at least one active pharmaceutical ingredient.

4. The metered dose inhaler of claim 1, wherein the metered dose inhaler delivers at least 1.0 mg of the at least one active pharmaceutical ingredient per actuation.

5. The metered dose inhaler of claim 1, wherein the at least one active pharmaceutical ingredient comprises a mast cell stabilizer, a receptor tyrosine kinase inhibitor, a beta-2 adrenergic receptor agonist, a steroid, or a combination thereof.

6. The metered dose inhaler of claim 1, wherein the at least one active pharmaceutical ingredient is a micronized crystalline solid.

7. The metered dose inhaler of claim 1, wherein the formulation is substantially free of ethanol.

8. The metered dose inhaler of claim 1, wherein the formulation includes at most 5% of ethanol by weight.

9. The metered dose inhaler of claim 1, wherein the actuator nozzle comprises an exit orifice diameter of at most 0.5 mm.

10. The metered dose inhaler of claim 1, wherein the formulation comprises at least 1% by weight of the at least one active pharmaceutical ingredient.

11. A metered dose inhaler comprising:
a metering valve;
a canister; and
an actuator comprising an actuator nozzle;
wherein the canister comprises a formulation, the formulation comprising greater than 70% by weight of HFA-152a; and
at least one active pharmaceutical ingredient suspended in the formulation;
wherein the metered dose inhaler delivers at least 0.5 milligram (mg) of the at least one active pharmaceutical ingredient per actuation.

12. The metered dose inhaler of claim 11, wherein the formulation comprises at least 95% by weight of HFA-152a.

13. The metered dose inhaler of claim 11, wherein the metered dose inhaler delivers a dose comprising at least 15% fine particle fraction of less than 5 μm in diameter of the at least one active pharmaceutical ingredient.

14. The metered dose inhaler of claim 11, wherein the metered dose inhaler delivers at least 1.0 mg of the at least one active pharmaceutical ingredient per actuation.

15. The metered dose inhaler of claim 11, wherein the at least one active pharmaceutical ingredient comprises a mast cell stabilizer, a receptor tyrosine kinase inhibitor, a beta-2 adrenergic receptor agonist, a steroid, or a combination thereof.

16. The metered dose inhaler of claim 11, wherein the at least one active pharmaceutical ingredient is a micronized crystalline solid.

17. The metered dose inhaler of claim 11, wherein the formulation is substantially free of ethanol.

18. The metered dose inhaler of claim 11, wherein the formulation includes at most 5% of ethanol by weight.

19. The metered dose inhaler of claim 11, wherein the actuator nozzle comprises an exit orifice diameter of at most 0.5 mm.

20. The metered dose inhaler of claim 11, wherein the formulation comprises at least 1% by weight of the at least one active pharmaceutical ingredient.
